(19) United States
(12) Patent Application Publication
(10) Pub. No.: US 2012/0114524 A1
(43) Pub. Date: May 10, 2012

(54) Surface Decontamination of Prefilled Containers in Secondary Packaging

(76) Inventor: Juergen Sigg, Loerrach (DE)

(21) Appl. No.: 13/382,380

(22) PCT Filed: Jul. 13, 2010
(86) PCT No.: PCT/EP10/60011

§ 371 Date: Jan. 5, 2012

(30) Foreign Application Priority Data
Jul. 14, 2009 (EP) 09165456.6

(51) Int. Cl. A61L 2/22; B01J 19/08

(52) U.S. Cl. 422/24; 422/22; 422/28; 422/33; 422/186.05; 422/243; 422/292

(57) ABSTRACT

Methods and systems for the terminal sterilization and surface decontamination of prefilled containers containing sensitive drug products, such as biotech drug products that are otherwise temperature or radiation sensitive, and thus not suitable for terminal sterilization by classical methods involving steam or gamma rays. The methods and systems are especially suited for prefilled containers in secondary packaging. Methods include terminal sterilization by exposing prefilled containers in secondary packaging to tunable-beta radiation and further include terminal sterilization by exposing prefilled containers to controllable vaporized-hydrogen peroxide, including application of measures to reduce or prevent diffusion of vaporized-hydrogen peroxide into prefilled containers.

### FIELD OF THE INVENTION

This invention relates to a method and system for terminal sterilization of the outer surface and/or surface decontamination of prefilled containers in secondary packaging, wherein the prefilled container contains a pharmaceutical or biological drug product.

### BACKGROUND

Prefilled containers are a type of medical device that are filled by the manufacturer at the time of assembly and provided to the end user, generally a health-care provider or a patient requiring treatment, in a sterile condition.

Prefilled containers offer several advantages over traditional packaging of therapeutics, including ease of use, reduced risk of contamination, elimination of dosing errors, increased drug supply and reduced waste. Of the various types of prefilled containers, prefilled syringes are the most common and best suited for parenteral administration of therapeutic products.

Various methods of sterilization of medical devices are known, but not all methods work with syringes, especially syringes prefilled with a drug or protein solution.

Steam sterilization is commonly employed for sterilizing medical devices, which typically involves heating the device in a steam autoclave. The heat and pressure generated in the autoclave, however, can have an adverse effect on the device and, more importantly, on the integrity of the drug product filled into the device. Steam sterilization may compromise the aesthetics of the product due to packaging degradation from high temperature steam treatment. Moreover, the high temperatures of the process (e.g. 120° C.-132° C.) preclude its use with heat sensitive materials, such as biotech drug products, specifically protein or other biological solutions.

Radiation exposure is also commonly employed for sterilizing medical devices, in which the product is subjected to ionizing radiation, such as gamma irradiation. Radiation exposure results in harmful damage to sensitive solutions, specifically causing destruction to sensitive biologicals such as proteins, as well as generation of massive amounts of peroxides in aqueous solutions that in a secondary reaction further may damage the active ingredient. Further, sterilizing doses of gamma rays cause a brown discoloration of glass parts of the device, and is prone to damage elastomeric materials like plunger stoppers. This destruction of the elastomers leads to increased stickiness of the components thus impairing the functionality of the system. Thus radiation is not an appropriate means for sterilizing prefilled containers, such as syringes, containing a biotech drug product.

Cold sterilization is a term collectively used for sterilization methods carried out at temperatures substantially below those of the steam process; attempts have been made to use ethylene oxide and hydrogen peroxide vapors as sterilants for this treatment. Treatment with sterilizing gasses, however, bears the risk of insufficient removal of the oxidizing gas. Diffusion of gas into the product container affects the stability of the drug product through chemical modification by gas vapors, such as alkylation and oxidation.

Prefilled syringes, although filled under aseptic conditions, are not packed into their secondary packaging in an aseptic environment and are therefore likely to be microbiologically contaminated at their outside. Terminal sterilization of prefilled containers in secondary packaging is one way to provide the device to an end user with a low bio-burden and low risk of contaminants, for safe application of the product by the end user. Moreover there is a strong market need for terminally antimicrobially-treated medical devices, such as prefilled syringes used for intravitreal injections.

Due to the sensitive nature of certain drug products, such as proteins, it is not possible to perform terminal sterilization and surface decontamination of containers filled with such products using current methods, like steam, irradiation or cold sterilization. Specifically, high temperatures are known to denature proteins and gamma radiation has been shown to chemically modify biological solutions. Radiation techniques, such as sterilization using gamma or beta radiation causes discoloring of packaging material and affects the long term stability of therapeutic agents such as protein or peptide solutions. As discussed above, oxidizing gases, while efficient for killing bacterial contamination, also harm biological molecules in sensitive therapeutic solutions.

As protein and biological molecules will be more and more developed for therapeutic use, the need for a terminal surface sterilization and surface decontamination method that is not harmful to the drug product will continually increase in the near future. Moreover, as regulatory agencies may require higher levels of sterility assurance, pharmaceutical and biotech companies will seek alternative procedures to approach or meet mandated-microbiological purity levels, without compromising the safety and efficacy of pharmaceutical preparations.

### SUMMARY

Described herein is a terminal sterilization and surface decontamination treatment of prefilled containers, specifically for sterilization of prefilled containers containing sensitive solutions, such as a drug product or biological therapeutic, within secondary packaging. In one embodiment, terminal sterilization is achieved by treating prefilled containers within secondary packaging with controllable vaporized-hydrogen peroxide (VHP). The principle is the formation a vapor of hydrogen peroxide in containment and a subsequent removal or inactivation of vapors in a controlled manner. Prior to removal or inactivation, VHP condenses on all surfaces, creating a microbicidal film that decontaminates the container surface.

It has been discovered that by varying the parameters of the antimicrobial treatment, for example—temperature, humidity, treatment duration, pressure, etc., conditions are generated that prevent the leaching of VHP into the syringes. As an example, the application of a vacuum at the end of the treatment will inverse the diffusion direction and reduce, if not stop, leaching of hydrogen peroxide through the rubbers. Further, inclusion of a gas plasma treatment after completion of the vaporized hydrogen peroxide cycle will further degrade all potentially remaining hydrogen peroxide residues. Prevention or reduction of leaching of detrimental concentrations of hydrogen peroxide into the protein solution in the syringe, either by removal of vapors or inactivation of vapors, ensures that the long-term stability of the protein is not compromised. It further has been found that among the commercially available primary packaging components, there are only very few packaging material combinations that provide the required tightness of the system such as to avoid ingress of sterilizing gasses into the pharmaceutical liquid enclosed by the prefilled container.

Further described herein is terminal sanitization or sterilization and surface decontamination of prefilled containers within secondary packaging by tunable electron beam (low-energy beta-ray) irradiation technologies as an alternative to aseptic inspection and aseptic secondary packaging operations.

In one embodiment, the use of low penetration depth radiation from a low-energy electron beam generator for a new application to sterilize the surface of secondary packaged drug product containers avoids aseptic packaging. In another embodiment, the penetration depth of electron beam radiation is tunable by adjustment of the accelerator voltage of the irradiation generator.

Generally, the concepts presented herein are applicable to all drug products having requirements or desirability for absence of viable organisms of the drug product container surface. The method and system described herein decontaminate or, more preferably render sterile an outside surface of primary packaged drug products within a secondary pack, thereby improving safety of products for critical administration (e.g. use in a surgical suite or for intravitreal injections).

The foregoing summary provides an exemplary overview of some aspects of the invention. It is not intended to be extensive, or absolutely require any key/critical elements of the invention.

### BRIEF DESCRIPTION OF THE DRAWINGS

The detailed description is explained with reference to the accompanying figures. In the figures, the left-most digit(s) of a reference number identifies the figure in which the reference number first appears.

FIG. 1 shows an exemplary prefilled container in secondary packaging that is decontaminated on surfaces according to the methods detailed herein.

FIG. 2 illustrates a block diagram of an exemplary system for surface decontamination of prefilled containers using vaporized-hydrogen peroxide.

FIG. 3 illustrates a block diagram of an exemplary system for surface decontamination of prefilled containers using tunable-beta radiation.

### DETAILED DESCRIPTION

The method and system described herein are for the sterilization and surface decontamination of prefilled containers containing sensitive solutions, such as drug products that are otherwise temperature or radiation sensitive or are sensitive to traces of oxidizing substances, and thus not suitable for terminal sterilization by classical methods involving steam, gamma or beta rays or sterilization with oxidizing gases or liquids. The method and system described herein are especially suited for prefilled containers that have been filled under aseptic conditions and been subject to additional processing, such as product labeling and subsequent secondary packaging. Methods include terminal sterilization and surface decontamination by exposing prefilled containers in secondary packaging to tunable-beta radiation and further include terminal sterilization and surface decontamination by exposing prefilled containers to controllable vaporized-hydrogen peroxide, including measures to reduce or prevent the diffusion of vaporized-hydrogen peroxide into prefilled containers. The methods also include an optional step of actively destroying any residual peroxide molecules, for example, by means of gas plasma.

### DEFINITIONS

In describing and claiming the terminal sterilization and surface decontamination method, the following terminology will be used in accordance with the definitions set forth below.

“Aseptic” conditions refer to conditions free of bacterial or microbial contamination.

“Administration” refers to the method of administering treatment to a subject or patient in need thereof, such as parenteral administration, intravenous administration and intravitreal administration.

“Beta irradiation” refers to sterilization methods using beta rays.

“Cold sterilization” refers to sterilization techniques employing chemical agents, gases, or irradiation. A requirement of cold sterilization is that the technique is carried out at temperatures below those used for steam sterilization, such as autoclavation.

“Container”, as used herein, is meant to include vials, syringes, bags, bottles, or other means useful for storage of medical treatments, such as drug products, whether in solid or liquid form, and other biological agents, such as peptides, proteins or recombinant biologicals, whether in solid or liquid form. Containers may be reusable or disposable, and may have a medical, veterinary or non-medical purpose.

“Prefilled container”, refers to a container, such as a syringe, that is filled with a solution at the time of assembly and packaging and is deliverable for use to an end user, such as a health care professional or a patient needing treatment. This term also refers to prefilled containers integrated into an administration device.

An “instruction” or “instructional material” includes a publication, a recording, a diagram, or any other medium of expression which can be used to communicate the usefulness of the method or system of the invention for its designated use. The instruction or instruction material may be presented together as part of the system or provided separately, or independently of the process, to an end user.

“Isolation”, as used herein refers to practices in pharmaceutical production, filling and packaging, wherein a clean, or sterile environment, is separated from a non-sterile environment to limit or prevent the introduction or spread or contamination of infectious agents, such as microorganisms.

“Medical device”, as used herein, refers to a device used for administering medical treatment and whose production or sale must, in part, comply with requirements, such as safety requirements, set forth by a government agency, such as the Food and Drug Administration.

“Solution” as used herein refers to the contents of a container like a vial or a prefilled syringe and includes solutions of biological therapeutics and drug products, protein products, peptide products, biological products, imaging solutions and aqueous solutions. Ideally, solutions are those that are temperature, oxidation or radiation sensitive due to the molecular make-up of the solution.

“Secondary packaging” refers to packaging enclosing the prefilled container, such as plastic wrapping, foil wrapping, paper wrapping or other suitable wrapping, such as blister packs.

“Terminal-antimicrobial-surface treatment” refers to sanitization or sterilization of an assembled container, such as a syringe filled with a solution that is in turn encased in secondary packaging. Terminal-antimicrobial treatment, or sterilization, allows a secondarily packaged prefilled container to be provided in sterile outside condition at its point of use.

“Vaporized-hydrogen peroxide” refers to hydrogen peroxide in vapor form capable of creating a microbicidal film on a surface, such as the surface of a container or packaging material.

The terms “sterilization”, “decontamination”, “sanitization”, “antimicrobial treatment” are used interchangeably herein.

“Sterility” as used herein is meant to refer to complete absence of microbial life as defined by a probability of nonsterility or a sterility assurance level (SAL). The required SAL for a given product is based on regulatory requirements. For example, required SALs for health care products are defined to be at least 10⁻⁶, i.e. a chance of less than 1:1 million of a non-sterile product for aseptically manufactured and terminally sterilized products, respectively.

Reference herein to “one embodiment” or “an embodiment” means that a particular feature, structure, operation or characteristic described in connection with the embodiment is included in at least one embodiment of the invention. Thus, the appearances of such phrases or formulations herein are not necessarily referring to the same embodiment. Furthermore, various particular features, structures, operations or characteristics may be combined in any suitable manner in one or more embodiments.

### Terminal Sterilization and Surface Decontamination of Prefilled Containers

Terminal sterilization is the process of sterilizing and/or decontaminating a final packaged product. In contrast, an aseptic packaging process requires individual product components to be sterilized separately and the final package assembled in a sterile environment. Terminal sterilization of a product provides greater assurance of sterility than an aseptic process. Terminal sterilization is also desired and provides a market advantage in some instances for the use of certain medical devices, such as the use of secondarily packaged prefilled syringes for intravitreal administration.

Described herein are terminal-sterilization methods suitable for prefilled containers containing sensitive products, such as biotech (biological) drug solutions, which can otherwise be compromised when using classical terminal sterilization processes, such as steam, gamma irradiation or cold sterilization processes currently used in pharmaceutical production and assembly lines. While reference is given to drug products, such as heat or radiation-sensitive drug solutions containing biologicals such as peptides or proteins, it will be understood by those skilled in the art that any suitable drug product that is considered a therapeutic agent, whether in solution or solid form, can be housed—or contained—in a prefilled container. Thus, the prefilled container itself is not drug specific.

It has now been discovered that treatment of prefilled containers in secondary packaging by an application of vaporized-hydrogen peroxide, in which vapors are controllable by certain post-treatment measures, and exposure to tunable-beta radiation, in which the depth of penetration of beta rays into secondary packaging are controllable, are ideal for surface decontamination of prefilled containers, yet not harmful to the stability or integrity of the contents of the prefilled container.

The methods and embodiments described herein are suitable for use in pharmaceutical production and packaging in isolation or outside of isolation. Furthermore, the methods described herein are adaptable to different container formats or types, with minimal incremental costs to production plant design. A system is also provided which allows for surface decontamination of prefilled containers in secondary packaging, as well as a kit comprising instructional material for practicing the method and system described herein.

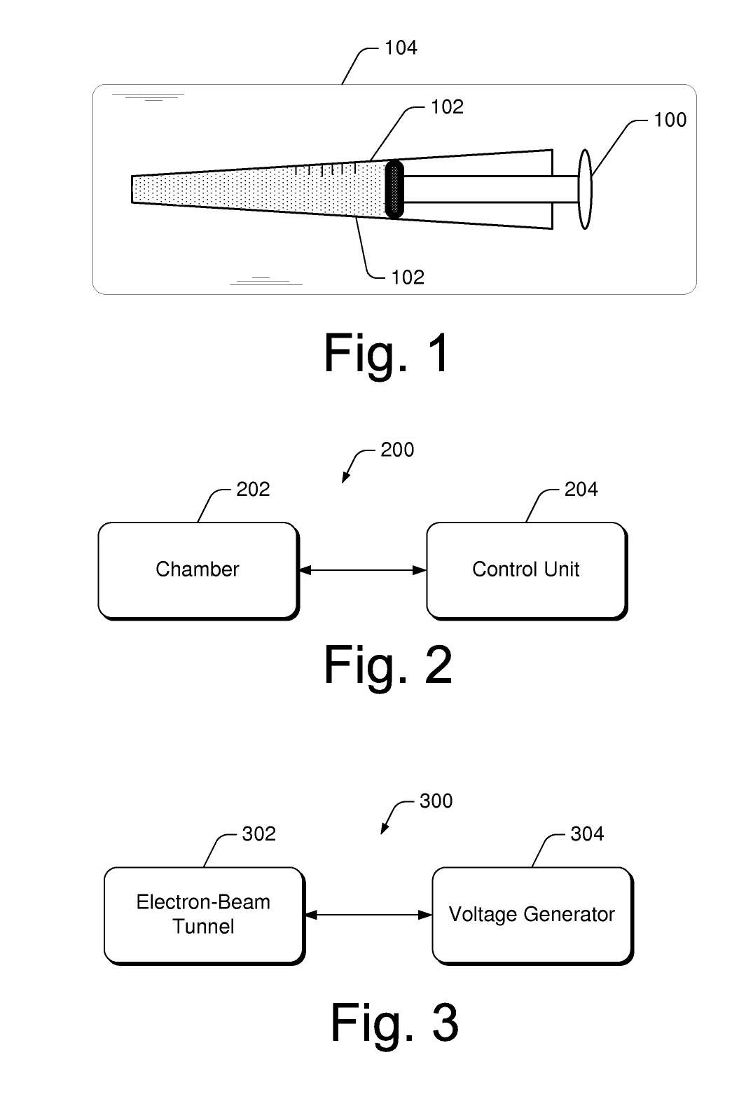

Referring to FIG. 1, a prefilled container 100 previously filled under aseptic conditions is decontaminated on surfaces 102 following encasement or packaging in a secondary package 104 by vaporized-hydrogen peroxide or tunable-beta radiation as described herein. FIG. 1 shows one exemplary prefilled container, however, it will be understood by those skilled in the art that various containers, other than a syringe, are also suitable. Moreover, while the exemplary container shown at FIG. 1 is a syringe in a closed and assembled position, it should be understood that other variants are envisioned. For example, a prefilled container not sealed by a stopper, plunger or other sealing mechanism can be surface decontaminated on interior portions of the container.

In one embodiment, the prefilled container is a syringe. Other suitable prefilled containers include vials, bottles, bags and other medical devices capable of containing a sterile solution or a solution requiring sterilization.

In one embodiment, the syringe is filled with a drug product, such as in the form of liquid, solution, powder or solid. In another embodiment the drug product is a solution such as a drug solution or protein solution that is otherwise sensitive to exposure to high temperatures, such as those used in steam sterilization, and ionizing energy, such as gamma or beta rays and oxidizing gasses. In yet another embodiment the drug product is one that has been lyophilized, in other words a solid, and requires reconstitution in liquid or solution prior to use.

In another embodiment, a solution is any drug product having requirements or desirability for sterility of the drug product container surface. In one particular embodiment, the drug product is a protein solution, such as ranibizumab (e.g. 6 mg/ml or 10 mg/ml) solution for intravitreal injection.

In one embodiment, the container is filled with solution under aseptic conditions, whether by an automated or manual process. Thus, the contents of the container are sterile and unaffected by surface decontamination methods as described herein. The term “filled” is meant to refer to the placement of contents, such as solution, into the container in an appropriate amount, such as an appropriate volume or appropriate concentration. The appropriate amount, volume or concentration will vary depending on the nature of the contents and their intended use.

In one embodiment, the container is considered a primary packaging for the solution contained within. In another embodiment, the prefilled container is packaged within a secondary package or packaging encasing the prefilled container. Suitable secondary packaging includes wrappings, such as paper, plastic or foil, and blister packs impermeable for microbes.

In one embodiment the prefilled container in secondary packaging undergoes decontamination, such that the contents of the secondary packaging, specifically the surfaces of the prefilled container, are decontaminated and terminally sterilized. Thus, prefilled container surfaces enclosed in a secondary packaging decontaminated by the methods described herein can be presented to, and opened within, a critical or sterile environment, such as a surgical suite.

In one embodiment, terminal sterilization and surface decontamination of prefilled containers within secondary packaging is carried out by treating surfaces of the prefilled container within secondary packaging with vaporized-hydrogen peroxide and applying post-treatment measures, within a decontamination chamber. A suitable decontamination chamber is any chamber, such as an autoclave, that has the means for reversibly sealing a closed environment and equipped with means of manipulating pressure, temperature, inflow and outflow of air within the chamber. Additional elements of a suitable chamber include the means for accommodating treatment by vaporized-hydrogen peroxide and post-treatment measures to reduce or prevent vaporized-hydrogen peroxide from entering into prefilled containers.

In another embodiment, the chamber is configured to accommodate the quantity of containers requiring terminal sterilization. Thus, in large-scale production and assembly lines, the chamber can be configured to accommodate a large quantity of containers, accordingly.

Treatment with vaporized-hydrogen peroxide is brought about by the application or release of hydrogen-peroxide-vapors within the decontamination chamber. In one embodiment, vapors of hydrogen peroxide are controllable, in other words, certain post-treatment measures are applied to manipulate or control the action of vaporized-hydrogen peroxide. In one embodiment, post-treatment measures are applied that direct—or reverse—the direction of vapor diffusion, such that vapors are prevented from entering into the prefilled container. In another embodiment, additionally post-treatment measures are applied that destroy any residual peroxide traces.

In one embodiment, post-treatment measures include reducing or eliminating gas radicals formed by action of vaporized-hydrogen peroxide. In yet another embodiment, post-treatment measures include inactivating vaporized-hydrogen peroxide action, such as oxidative action.

In another embodiment, terminal sterilization and surface decontamination of prefilled containers within secondary packaging is achieved by application of tunable beta ray irradiation. In one embodiment, the surface of a prefilled container in secondary packaging is decontaminated by an adjustment of accelerator voltage of an irradiation generator to provide beta radiation of a sufficient dose to penetrate secondary packaging without penetrating primary packaging.

In another embodiment, the accelerator voltage required to deliver the appropriate amount of beta radiation to decontaminate the surface of prefilled containers depends on the thickness of secondary packaging materials. For example, in one embodiment, suitable packaging materials are less than or equal to 0.05 mm in thickness. Such materials of less than or equal to 0.05 mm in thickness may be made of foils.

In another embodiment a combination of secondary and primary packaging components, accelerator voltage, irradiation plant design and throughput speed allow surface decontamination of a prefilled container in secondary packaging, while almost completely shielding contents of the prefilled container by primary packaging materials.

In one embodiment, a suitable primary packaging is a syringe capable of shielding irradiation sensitive solution contained within. Shielding can be provided by the thickness of the container walls or the material components of the container. Shielding effectiveness can be determined by adjustment of the accelerator voltage and thus the depth of penetration of the beta rays emitted onto the prefilled container. Furthermore, shielding is determined by measuring the absorbed dosage, such as with a dosimeter.

It is understood by those in the art that a prefilled container is assembled under aseptic conditions, such that the contents of the container are sterile. While contents of the container are sterile, the surface of the container is susceptible to contamination during further packaging and product labeling using standard pharmaceutical packaging protocols. For surface decontamination of prefilled containers, the sterilization methods herein are adaptable to standard production and packaging of pharmaceutical products in isolation or outside of isolation.

In one embodiment, a prefilled container previously filled under aseptic conditions and labeled and packaged into secondary packaging by a manual or automated process is presented to an electron beam tunnel for terminal sterilization and surface decontamination of the final packaged product. In one embodiment, the prefilled container in secondary packaging is introduced, either by a manual process or automated process, or a combination of the two, into the electron beam tunnel via an inlet and transported for all or a portion of time through the e-beam tunnel to an outlet as the surfaces of prefilled containers in secondary packaging are exposed to low-energy beta radiation. In another embodiment, prefilled containers in secondary packaging remain stationary for all or a portion of time as the surfaces of prefilled containers in secondary packaging are exposed to low-energy beta radiation. In another embodiment, the electron beams are oscillated, e.g. by application of magnetic fields, such that the whole surface of the object is scanned by the electron beam. In another embodiment, the object is passed below the scanning electron beams by means of a transport mechanism like a moving conveyor. In another embodiment, the chamber for electron beam treatment is open, but shielded to the environment by a tortuous path of the objects into and out of the chamber.

### Terminal Sterilization of Prefilled Container by Vaporized-Hydrogen Peroxide (VHP)

In one embodiment, terminal sterilization of prefilled containers in secondary packaging is carried out by antimicrobial treatment in a chamber with vaporized-hydrogen peroxide, also referred to as “cold sterilization”.

The various steps, or operations, involved in the sterilization and surface decontamination process can be performed automatically under the administration of a system manager, such as a microprocessor. Alternatively, operations can be performed separately in manual operations. Furthermore, operations can be performed in a combination of automated and manual processes.

In one embodiment prefilled containers are enclosed in secondary packaging following filling of containers under aseptic conditions. In another embodiment, prefilled containers are labeled with any product information, such as product name, indications; use instructions, etc., prior to encasement of prefilled containers in secondary packaging.

In one embodiment, prefilled containers in secondary packaging are presented either manually or automatically to, and secured within, a decontamination chamber.

A suitable decontamination chamber is any chamber, such as an autoclave, equipped with means for reversibly sealing a closed environment, and equipped with means of manipulating pressure, temperature, inflow and outflow of air within the chamber. Additional elements of a suitable chamber include means for accommodating treatment by VHP and post-treatment measures to reduce or prevent VHP from entering into prefilled containers. A further element of a suitable chamber is means to destroy any remaining peroxide traces.

In one embodiment, hydrogen peroxide vapor is introduced into the chamber, either generated within or released within the chamber for a sufficient time to decontaminate—or treat—the surface of prefilled containers in secondary packaging. In another embodiment, application of vaporized-hydrogen peroxide is carried out at temperatures below those used for steam sterilization.

Hydrogen peroxide in liquid form has long been recognized as a disinfectant. Koubek U.S. Pat. No. 4,512,951 describes a method of sterilization with liquid hydrogen peroxide which includes vaporizing an aqueous solution of hydrogen peroxide and passing the resulting hydrogen peroxide-water vapor mixture into an evacuated sterilization chamber where, upon contact with items to be sterilized, the vapor condenses to form a layer of liquid hydrogen peroxide on the items. The items to be sterilized are maintained at a temperature below the dew point of the hydrogen peroxide-water mixture to assure condensation, but the overall chamber temperature must be high enough to prevent condensation of the incoming vapor before it reaches the items. Following a suitable time for sterilization, the condensate is revaporized by passing filtered, preferably heated air over the surface of the items. Sterilization with gaseous hydrogen peroxide is described by Moore et al. U.S. Pat. No. 4,169,123 and Forstrom et al. U.S. Pat. No. 4,169,124. The methods described in those two patents involve surrounding an article to be sterilized with vapor phase hydrogen peroxide and maintaining contact between the article and the sterilant at temperatures below 80° C. until sterility is achieved. The lowest temperature disclosed in either the Moore or Forstrom patents is 20° C.

It has been determined that with sensitive solutions, such as protein solutions, leaching of vaporized-hydrogen peroxide into the prefilled container is detrimental to the molecular integrity of the solutions because hydrogen peroxide vapors that enter the container cause chemical modifications of the solution, such as oxidation.

It has now been discovered that applying post-treatment, or post-application, measures reduces or prevents the adverse effects of VHP on sensitive solutions and preserve the integrity, and thereby therapeutic efficacy, of otherwise sensitive solutions in prefilled containers. Post-application measures are ideally those measures that deactivate the oxidizing action of hydrogen peroxide, whether by removing vaporized-hydrogen peroxide or rendering hydrogen peroxide vapors into an inactive state.

In one embodiment, leaching of VHP into a prefilled container is prevented by application of a vacuum at the end of the antimicrobial treatment in the chamber to inverse the diffusion direction of hydrogen peroxide vapors. By reversing the direction of vapor flow, hydrogen peroxide vapors are prevented from entering the prefilled container, thereby maintaining the integrity of the sensitive solution within the container while the surface of the container is decontaminated.

In yet another embodiment, hydrogen peroxide vapors are inactivated, such that they are incapable of chemically modifying the solution contained in a prefilled container. In another embodiment, post-treatment measures include neutralizing the oxidative ability of hydrogen peroxide vapors. In yet another embodiment, hydrogen peroxide vapors are inactivated by application of ultraviolet rays to the container after a sufficient exposure time of prefilled container to VHP following treatment. Other suitable inactivating agents, such as chemical agents or gas plasma, can be applied post-treatment to inactivate VHP following a sufficient exposure time of the surfaces of prefilled containers to VHP.

At the conclusion of the terminal sterilization process, the prefilled container in secondary packaging may be removed from the chamber, and is suitable for use by an end user.

In one embodiment, the sterilization process may be performed by an automated system. For example, referring to FIG. 2, illustrated is a block diagram of a system 200 for decontaminating a surface of a prefilled container in secondary packaging. System 200 includes a sealed chamber 202 and a control unit 204 coupled, directly or indirectly, to the chamber 202.

In one embodiment, the sealed chamber 202 may be any suitable decontamination chamber. For instance, the chamber 202 may include an autoclave, with the ability to reversibly seal a closed environment. The chamber 202 may also be equipped with mechanisms to manipulate pressure, temperature, and inflow and outflow of air within the chamber 202.

Control unit 204 provides instructions, in the form of signals, to chamber 202 to perform operations associated with sterilizing a prefilled container 100 (such as shown in FIG. 1) in a prescribed-automatic manner. Control unit 204 may transmit signals to chamber 202 to direct chamber 202 (or related parts) to physically enable a vaporized-hydrogen peroxide to come into contact the surface of the prefilled container in the secondary packaging.

For example, in one embodiment, the control unit 204 may transmit a signal to a valve (not shown) associated with a reservoir for passing vaporized-hydrogen peroxide into the chamber. The control unit 204 measures a preset duration-of-time the vaporized-hydrogen peroxide is to remain in contact with the prefilled-container surface. Upon expiration of the preset duration-of-time, the control unit 204 transmits a signal to chamber 202 (or a related device) to cause a post-decontamination measure to occur to reduce the presence of vaporized-hydrogen peroxide in the chamber, thereby preventing vaporized-hydrogen peroxide from diffusing into the prefilled container undergoing surface decontamination.

For example, following surface decontamination, the control unit 204 may transmit a signal to a vacuum (not shown) to reverse the flow of hydrogen-peroxide vapors out of the chamber 202 to remove these vapors from the chamber. Other suitable control mechanisms for controlling hydrogen-peroxide vapors include mechanisms for introducing neutralizing or inactivating agents, such as chemical agents, into the chamber 202, which upon contact with hydrogen-peroxide vapors render the vapors inactive, and thus harmless to the interior solution of a prefilled container.

Reference is made to treatment times that are sufficient to terminally sterilize the prefilled container. In one embodiment, a sufficient treatment time or the duration of the presence of vaporized-hydrogen peroxide within the chamber to sufficiently decontaminate the container surface is determined by routine validation. For example, containers that have been subjected to treatment by vaporized-hydrogen peroxide are compared to controls and can be checked for bacterial contamination using standard laboratory protocols, such as incubation of suspected contaminated object with bacterial growth medium and then checking for bacterial growth, generally performed by the use of bioindicators. By plotting treatment time against presence of bacterial growth, the treatment time to achieve decontamination, thus the absence of bacterial growth, can easily be determined. Validation techniques apply whether terminal sterilization is carried out by vaporized-hydrogen peroxide as described above or carried out by exposure to beta radiation as described below.

In one embodiment, the control unit 204 is automated, and operates in accordance with code executing on a processor. The implementation of a control unit will be well within the scope of someone skilled in the art. For instance, the control unit may be any personal computer, microprocessor, or other suitable devices, capable of executing code that is programmed to transmit signals to devices associated with physically carrying out the sterilization process.

It will be appreciated that the various steps, or operations, involved in the sterilization and surface decontamination process can be performed automatically under the administration of a control unit as described above. Alternatively, operations can be performed separately in manual operations. Furthermore, operations can be performed in a combination of automated and manual processes.

### Terminal Sterilization of Prefilled Containers by Tunable-Beta Irradiation

In one embodiment, terminal sterilization of prefilled containers in secondary packaging is carried out by a decontamination treatment in a chamber equipped with one or more electron beam generators that are tunable to generate an appropriate dose of beta radiation onto the surfaces of the prefilled containers.

The various steps, or operations, involved in the sterilization and surface decontamination process can be performed automatically under the administration of a system manager, such as a microprocessor. Alternatively, operations can be performed separately in manual operations. Furthermore, operations can be performed in a combination of automated and manual processes.

In one embodiment prefilled containers are enclosed in secondary packaging following filling of containers under aseptic conditions. In another embodiment, prefilled containers are labeled with any product information, such as product name, indications; use instructions, etc, prior to encasement of prefilled containers in secondary packaging.

In one embodiment, prefilled containers in secondary packaging are presented either manually or automatically to a decontamination chamber with an inlet side and an outlet side. In another embodiment the decontamination chamber is an electron beam tunnel. In yet another embodiment, prefilled containers are mechanically moved through the tunnel from the inlet side to the outlet side on a movable mechanism, such as a conveyor. Thus, prefilled containers move through the chamber as the surfaces of prefilled containers are exposed to beta irradiation.

In another embodiment, the electron beams are oscillated, e.g. by application of magnetic fields, such that the whole surface of the object is scanned by the electron beam. In another embodiment, the object is passed below the scanning electron beams by means of a transport mechanism like a moving conveyor.

In one embodiment, the surfaces of prefilled containers in secondary packaging are decontaminated during an exposure time of low penetration beta radiation of less than one second, ideally in less than one-half second. Thus, treatment times with tunable-beta radiation as described herein are significantly less than decontamination using gamma rays, which require surface treatment times of several hours or longer for sufficient decontamination and sterilization.

In another embodiment, the electron beam tunnel is configured with an electron beam generator, whereby the voltage of energy generated is tunable.

In yet another embodiment, prefilled containers in secondary packaging are transported or moved about in a fashion as to expose all surfaces of the containers to emitted beta radiation within the tunnel.

Primary packaging containers for sterile pharmaceutical drug products are often up to about 30-fold thicker than the secondary packaging material. In one embodiment the thickness of the wall of the primary packaging material is 20 or more times thicker than the thickness of the secondary packaging material, thus allowing a resulting dose absorbed by the contents in the prefilled container to less than 0.1 kGy.

It has been discovered that it is possible to find a combination of packaging components, accelerator voltage, irradiation plant design and throughput speed that allow a surface decontamination or surface sterilization of a prefilled container in secondary packaging, while the contents of the container are essentially shielded by the primary packaging material. Therefore, beta irradiation does not affect sensitive biomolecules, such as biotech drug solutions, inside the primary packaging materials.

In one embodiment, beta irradiation of the prefilled container may be conducted at any dosage useful to provide effective sterilization without degrading the container or its contents, using any known beta irradiation apparatus, such as a low voltage generator or particle accelerator, with the amount of radiation depending on the thickness of the secondary packaging

In one embodiment the minimum sterilizing dose (MSD) of beta radiation is that required to deliver the required SAL for the product. In one embodiment sterilizing doses are measured with Gray (Gy) or Rad (radiation absorbed dose). In another embodiment, absorbed doses are measured by dosimeter, preferably by film dosimeters, calorimeters or cerium dosimeters.

In another embodiment, the amount of radiation depends on the presence of secondary packaging and the thickness of the secondary packaging. For a typical prefilled container, the beta radiation is desirably provided at a dosage of 25 kGy at the surface of the prefilled container.

In one embodiment, a particle accelerator generates beta-particle acceleration through a vacuum tube. In one embodiment, acceleration is by means such as magnetic field, electrostatic charge or by energy transfer from high frequency electromagnetic waves.

At the conclusion of the terminal sterilization process, the prefilled container in secondary packaging leaves the tunnel by the outlet with surfaces decontaminated and is suitable for use by an end user. Because treatment time for surface decontamination is as short as about one second, surface decontamination of prefilled containers in secondary packaging offers numerous advantages over sterilization methods involving gamma radiation, which are harmful to container contents, require significantly longer exposure times for decontamination, and require additional shielding along the production line, and cause discoloration of packaging components, Moreover, sterilization techniques involving gamma radiation cause significant bottlenecks in production assembly lines which are eliminated by surface decontamination using tunable-beta radiation in an e-beam tunnel.

In one embodiment, as depicted in FIG. 3, a system 300—for surface-decontaminating a prefilled container in secondary packaging—includes an electron-beam tunnel 302 equipped with one or more tunable-electron beam generators, shown as voltage generators 304. In another embodiment, the one or more tunable-electron-beam generators 304 of the system are configured to variably generate low-energy beta radiation. Alternatively, electron beams are oscillated, such that the electron beams hit a larger surface of a prefilled container and increase the exposure surface of the container.

In yet another embodiment, the one or more generators 304 apply an accelerator voltage to produce a sufficient amount of beta radiation to decontaminate the surface of the prefilled container, wherein the sufficient amount of beta radiation depends on the thickness of the secondary package and the thickness of the prefilled container. Thus, beta radiation is allowed to penetrate the secondary package while the thickness of the prefilled container shields the contents therein from beta radiation.

Reference is made to treatment times that are sufficient to terminally sterilize and surface decontaminate the prefilled container. In one embodiment, a sufficient treatment time or the duration of the presence of low-energy beta radiation within the tunnel to sufficiently decontaminate the container surface is determined by routine validation. For example, containers that have been subjected to treatment by beta radiation are compared to controls and can be checked for bacterial contamination using standard laboratory protocols, such as incubation of suspected contaminated object with bacterial growth medium and then checking for bacterial growth. By plotting treatment time against presence of bacterial growth, the treatment time to achieve decontamination, thus the absence of bacterial growth, can easily be determined. Validation techniques apply whether terminal sterilization is carried out by beta radiation as described above or carried out by exposure to VHP as described above.

Reference is now made to the following examples. These examples are provided for the purpose of illustration only and should in no way be construed as being limited to these examples but rather should be construed to encompass any and all variations, which become evident as a result of the teaching provided herein.

### Example 1

In the following experiment, prefilled syringes were treated with a vaporized-hydrogen peroxide sterilization treatment in a chamber, either by a single pass through a VHP sterilization procedure or two passes (shown in the table below as 2×) through a VHP sterilization procedure. Syringes containing protein solutions treated by VHP were compared to control syringes treated with VHP to determine if the integrity of proteins present in solution was maintained.

A formulation as described in U.S. Pat. No. 7,060,269 was tested for protein degradation following treatment by VHP.

Approximately 10 mL of solution was filtered through a 0.22 μm syringe filter. (Millex GV filter available from Millipore, Billerica, Mass. USA.) Filling of 0.5 mL syringes was performed in a sterile lab for hydrogen peroxide treatment.

Analysis after the treatment with VHP revealed the following protein contents, visualized by HPLC analysis: byproducts and degradation products by HPLC (IEC) and by-products and degradation products by HPLC (SEC).

TABLE 1
Protein Stability Following Treatment with VHP

| | IEC | IEC | SEC |
|---|---|---|---|
| Batch | (% main peak) | (% basic peak) | (% monomer) |
| Control | | | |
| 9823.01 CSi | 98 | 2 | 100 |
| 9823.02 CSi | 98 | 2 | 100 |
| 1 × treatment | | | |
| 9823.04 CSi | 98 | 2 | 100 |
| 9823.05 CSi | 98 | 2 | 100 |
| 2 × treatment | | | |
| 9823.07 | 98 | 2 | 100 |
| 9823.08 | 98 | 2 | 100 |

The results seen were within the requirement; there were no differences between the results of the untreated syringes and with hydrogen-peroxide treated syringes. Analysis can also be carried out at different time points following treatment, such as 1 month, 3 months and six months following treatment by VHP, or over the shelf-life of the product of the prefilled container. Analysis can be carried out to determine continued stability of the protein solution, including tests by HPLC for presence of by-products using standard HPLC laboratory protocols. Analysis can also be carried out by the presence of physical changes, such as measuring the concentration of H₂O₂ in solution by a fluorescence test using an over-the-counter commercially available kit in conjunction with an apparatus with fluorescence detection.

### Example 2

The following experiment was carried out to determine the effectiveness of surface decontamination using beta irradiation. A commercially available e-beam tunnel for outside decontamination of containers, equipped with KeVAC accelerators from Linac Technologies (Orsay, France), was used to investigate the penetration depth of the electron beam in different materials. For example, penetration was measured in a polyethylene bag with foil thickness of 50 μm, an aluminum bag with foil thickness of 0.1 mm and a glass slide of 1 mm thickness.

To increase sensitivity of the study, multiple passes of the samples through the tunnel were investigated. Far West 60 Film dosimeters, available from Far West Technologies (Santa Barbara, Calif., USA) were used to record the radiation absorbed.

TABLE 2
Beta Irradiation Absorption by Packaging Materials:

| Number of passes | Absorbed dose | | | | |
|---|---|---|---|---|---|
| through | Dosimeter in | | Dosimeter in | | Dosimeter |
| decontamination | Polyethylene | | aluminum | | shielded by 1 |
| tunnel | bag | | bag | | mm glass slide |
| 1 pass | 30 | kGy | 1.3 | kGy | <LOQ(0.1 kGy) |
| 3 passes | 97 | kGy | 64 | kGy | <LOQ(0.1 kGy) |
| 5 passes | 207 | kGy | 105 | kGy | <LOQ(0.1 kGy) |

The feasibility study showed that already with these not optimized settings of the electron beam decontamination tunnel a surface sterilization could be obtained (>=25 kGy) when the product was packaged into plastic bags. Even after 5 times passing through the electron beam treatment tunnel, the absorbed dose within the packaging material (behind a 1 mm thick glass wall) was far below the limit of quantitation which was 1 kGy for the dosimeters used.

Additionally, the oxidative stress exerted on a 0.5% Polysorbate 20 solution in prefilled glass syringes (1 mL long, ISO) was investigated by measurement of peroxides according to standard protocols. The total amount of peroxides was measured by the Ferrous Oxide Oxidation (FOX) test, according to a standard protocol.

TABLE 3
Peroxide Levels Following Beta Irradiation of Prefilled Containers:

| Number of passes | Peroxide content of 0.5% Polysorbate |
|---|---|
| through E-beam | 20 solution in 1 mL long glass |
| tunnelin water | syringe (ISO) [μMol/mL] |
| Reference (not treated) | 0.04 |
| 1 pass | 0.04 |
| 3 passes | 0.03 |
| 5 passes | 0.05 |

No significant influence of the electron beam treatment on the peroxide content of the solution enclosed in glass syringes could be observed. Thus, beta irradiation proved safe to solutions within prefilled containers.

Additionally, the oxidative stress exerted on protein solution in prefilled glass vials was investigated by measurement of degradation products according to standard protocols.

A formulation as described in U.S. Pat. No. 7,060,269 was tested for protein degradation following treatment by electron beam irradiation. Approximately 0.3 mL of solution was filtered through a 0.22 μm filter and aseptically filled into pre-sterilized glass vials, aseptically closed with a sterile rubber stopper and secured with an aluminum crimp cap.

The containers were passed through the above described e-beam tunnel with identical settings as for the other experiments mentioned above. Containers were analyzed after the treatment with electron beam radiation to determine protein contents, visualized by HPLC analysis for byproducts and degradation products by HPLC (IEC), as performed above in Example 1.

TABLE 4
Protein Stability Following Beta Irradiation of Prefilled Containers

| Number of | | |
|---|---|---|
| passes through | | |
| E-beam tunnel | IEC (% main peak) | IEC (% basic peak) |
| Reference | 98 (97.8) | 1 (1.2) |
| (not treated) | | |
| 1 pass | 98 (97.8) | 1 (1.3) |
| 3 passes | 98 (97.5) | 2 (1.5) |
| 5 passes | 98 (97.6) | 1 (1.4) |

There were no differences between the results of the untreated syringes and with electron beam sterilized vials, following 1 pass, 3 passes or 5 passes through the e-beam sanitization process, as shown in the results at Table 4. Thus, tunable-beta radiation as described herein proved safe to solutions within prefilled containers.

The described embodiments are to be considered in all respects only as exemplary and not restrictive. The scope of the invention is, therefore, indicated by the subjoined claims rather than by the foregoing description. All changes which come within the meaning and range of equivalency of the claims are to be embraced within their scope.

1. A method for surface decontamination of a prefilled container in secondary packaging, comprising:
applying vaporized-hydrogen peroxide to the surface of the prefilled container in secondary packaging;
allowing vaporized-hydrogen peroxide to remain in contact with the prefilled container surface for a sufficient time to decontaminate the prefilled container surface; and
causing a post-decontamination measure to occur to reduce the presence of vaporized-hydrogen peroxide, thereby preventing vaporized-hydrogen peroxide from diffusing into the prefilled container, wherein the prefilled container contains a drug product otherwise sensitive to sterilization treatment by gamma radiation, sterilization treatment by exposure to steam, and sterilization treatment by exposure to vaporizing agents and gases.

2. The method of claim 1, wherein the prefilled container is a syringe containing a drug product otherwise sensitive to sterilization treatment by gamma radiation, sterilization treatment by exposure to steam, and sterilization treatment by exposure to vaporizing agents and gases.

3. The method of claim 1, wherein the prefilled container is a syringe containing a therapeutically effective amount of ranibizumab.

4. The method of claim 1, wherein sufficient time to decontaminate the surface of the prefilled container is determined by validation of treatment times and compared to a control standard.

5. The method of claim 1, wherein the post-decontamination measure includes applying a vacuum following the duration of treatment with vaporized-hydrogen peroxide, thereby reversing the direction of diffusion of vaporized-hydrogen peroxide and preventing intrusion of vaporized-hydrogen peroxide into the prefilled container.

6. The method of claim 1, wherein the post-decontamination measure includes applying ultraviolet rays following the duration of treatment with vaporized-hydrogen peroxide, thereby inactivating oxidative action of hydrogen peroxide vapors.

7. The method of claim 1, wherein the post-decontamination measure includes gas plasma treatment.

8. A method for surface decontamination of a prefilled container in secondary packaging, comprising:
presenting a prefilled container in a secondary package to an electron beam tunnel equipped with one or more tunable electron beam generators capable of variably generating low-energy beta radiation, and capable of oscillating electron beams such that a larger surface of the prefilled container is exposed to beta radiation during decontamination; and
applying an accelerator voltage of the one or more tunable electron beam generators to produce a sufficient amount of beta radiation to decontaminate the surface of the prefilled container, wherein the sufficient amount of beta radiation depends on the thickness of the secondary package and the thickness of the prefilled container, such that beta radiation is allowed to penetrate the secondary package while the thickness of the prefilled container shields the contents therein from beta radiation.

9. The method of claim 8, wherein the thickness of the wall of the primary packaging material is 20 or more times thicker than the thickness of the secondary packaging material, thus reducing the dose absorbed by the product in the container to less than 0.1 kGy.

10. The method of claim 8, wherein the prefilled container is a vial filled with a solution or solid otherwise sensitive to sterilization treatment by gamma radiation, sterilization treatment by exposure to steam, and sterilization treatment by exposure to vaporizing agents, gases or peroxide forming substances.

11. The method of claim 8, wherein the prefilled container is a syringe filled with a solution otherwise sensitive to sterilization treatment by gamma radiation, sterilization treatment by exposure to steam, and sterilization treatment by exposure to vaporizing agents and gases or peroxide forming substances.

12. The method of claim 8 wherein the prefilled container is a syringe containing a therapeutically effective amount of ranibizumab.

13. The method of claim 8, wherein the penetration depth is measured by dosimetry.

14. The method of claim 8, wherein sufficient energy to decontaminate a surface of a prefilled container is that which provides a dose of beta radiation of at least approximately 25 kGy to the container surface.

15. The method of claim 8, wherein sufficient energy to decontaminate a surface of a prefilled container is that which provides a dose of beta radiation yielding a 10⁻⁶ Sterility Assurance Level of the outside of the container surface.

16. A system for decontaminating a surface of a prefilled container in secondary packaging, the system comprising:
a sealed chamber; and
a control unit coupled to the chamber, the control unit configured to automatically (i) enable a vaporized-hydrogen peroxide to contact the surface of the prefilled container in the secondary packaging; (ii) allow the vaporized-hydrogen peroxide to remain in contact with the prefilled-container surface for a predetermined time; and (iii) cause a post-decontamination measure to occur to reduce the presence of vaporized-hydrogen peroxide in the chamber, thereby preventing vaporized-hydrogen peroxide from diffusing into the prefilled container, wherein the prefilled container contains a drug product otherwise sensitive to sterilization treatment by gamma radiation, sterilization treatment by exposure to steam, and sterilization treatment by exposure to vaporizing agents and gases.

17. A system for surface-decontaminating a prefilled container in secondary packaging, the system comprising: an electron-beam tunnel equipped with one or more tunable-electron beam generators, the tunable-electron-beam generators, configured to (i) variably generate low-energy beta radiation, (ii) oscillate the electron beams such that a larger surface of a prefilled container is exposed to electron beams; and (iii) apply an accelerator voltage to produce a sufficient amount of beta radiation to decontaminate the surface of the prefilled container, wherein the sufficient amount of beta radiation depends on the thickness of the secondary package and the thickness of the prefilled container, such that beta radiation is allowed to penetrate the secondary package while the thickness of the prefilled container shields the contents therein from beta radiation.

18. A kit for decontaminating the surface of a prefilled container in secondary packaging in a sealed chamber, the kit comprising: an instruction for using the sealed chamber to (i) apply a vaporized-hydrogen peroxide to contact the surface of the prefilled container in the secondary packaging; (ii) allow the vaporized-hydrogen peroxide to remain in contact with the prefilled-container surface for a predetermined time within the sealed chamber; and (iii) cause a post-decontamination measure to occur to reduce the presence of vaporized-hydrogen peroxide in the chamber, thereby preventing vaporized-hydrogen peroxide from diffusing into the prefilled container.

19. A kit for surface-decontaminating a prefilled container in secondary packaging, the kit comprising: an instruction for (i) variably generating low-energy beta radiation to contact the surface of the prefilled container; and (ii) produce a sufficient amount of beta radiation to decontaminate the surface of the prefilled container, wherein the sufficient amount of beta radiation depends on the thickness of the secondary package and the thickness of the prefilled container such that beta radiation is allowed to penetrate the secondary package while the thickness of the prefilled container shields the contents therein from beta radiation.

20. A system according to claim 16, wherein post-decontamination measure includes gas plasma treatment.

21. A kit according to claim 19, wherein post-decontamination measure includes gas plasma treatment.

22. The method of claim 1, wherein the drug product is a protein solution.
